SEQUENCE LISTING (11 sequences)

SEQ ID NO: 1 - DNA, 36 bp, Artificial Sequence
  Description of Artificial Sequence Synthetic Primer
taatacgactcactataggaattgcgggaaaggggt

SEQ ID NO: 2 - DNA, 36 bp, Artificial Sequence
  Description of Artificial Sequence Synthetic Primer
cgggcggaagacgcgccctgaactgcatccatatca

SEQ ID NO: 3 - DNA, 30 bp, Artificial Sequence
  Description of Artificial Sequence Synthetic Primer
gcgcgcgaattctaatacgactcactatag

SEQ ID NO: 4 - DNA, 21 bp, Artificial Sequence
  Description of Artificial Sequence Synthetic Primer
ccgcgggcggaagacgcgccc

SEQ ID NO: 5 - DNA, 307 bp, Artificial Sequence
  Description of Artificial Sequence Synthetic DNA Construct
gccagtgaattctaatacgactcactatagggtcgctcaaacattacctggtgttgagcgaaaagaatctcgaagacaagggcggcatggtcccagcctcctcgctggcgccgcctgggcaacatgcttcggcatggtgaatgggacctctagactgtgcatcgggtcaggactgaaaggtagcagccctgggcagttttttgaagtgcatcgggtcaggaccttcgggtagcagccctgggcaggatccctagcataaccccttggggcctctaaacgggtcttgaggggttttttgaagcttggc

SEQ ID NO: 6 - DNA, 306 bp, Artificial Sequence
  Description of Artificial Sequence Synthetic DNA Construct
gccagtgaattctaatacgactcactatagggtcgctcaaacattaagtggtgttgtgcgaaaagaatctcgaagacaagccggccatggtcccagcctcctcgctggcggccggtggcaacatgcttcggcatggtgaatgggacctctagactgtgcatcgggtcaggactgaaaggtagcagccctgggcagttttttcctgtgcatcgggtcaggaccttcgggtagcagccctgggcaggatccctagcataaccccttggggcctctaaacgggtcttgaggggttttttgaagcttggc

SEQ ID NO: 7 - DNA, 180 bp, Artificial Sequence
  Description of Combined DNA/RNA Molecule Synthetic DNA Construct
  Description of Artificial Sequence Synthetic DNA Construct
ctcgaagacaagccggccauggucccagccuccucgcgccggccggugggcaacaucguucgcgauggugaaugggaccucuagacugugcaucgggucaggacugaaagguagcagcccugggcaguuuuuuccugugcaucgggucaggaccuucggguagcagcccugggcaggauc

SEQ ID NO: 8 - RNA, 68 nt, Hepatitis D virus
gggcggcauggucccagccuccucgcuggcgccgccuggcaacaugcuucggcauggugaaugggacc

SEQ ID NO: 9 - RNA, 68 nt, Hepatitis D virus
gccggccauggucccagccuccucgcuggcggccgguggcaacaugcuucggcauggugaaugggacc

SEQ ID NO: 10 - RNA, 62 nt, Hepatitis D virus
cauggucccagccuccucgcuggcgccgccuggcaacaugcuucggcauggugaaugggacc

SEQ ID NO: 11 - RNA, 62 nt, Hepatitis D virus
cauggucccagccuccucgcuggcggccgguggcaacaugcuucggcauggugaaugggacc

(19) United States
(12) Patent Application Publication
(10) Pub. No.: US 2007/0249820 A1
(43) Pub. Date: Oct. 25, 2007

(54) Activatable Ribozymal Purification Constructs and Methods of Use

(75) Inventors: Robert T. Batey, Boulder, CO (US); Jeffrey S. Kieft, Denver, CO (US)

Correspondence Address: PAUL, HASTINGS, JANOFSKY & WALKER LLP, P.O. BOX 919092, SAN DIEGO, CA, 92191-9092, US

(73) Assignees: The Regents of the University of Colorado, Boulder, CO (US); University of Colorado, Boulder, CO (US)

(21) Appl. No.: 11/428,779

(22) Filed: Jul. 5, 2006

Related U.S. Application Data
(63) Continuation of application No. PCT/US05/00627, filed on Jan. 7, 2005.
(60) Provisional application No. 60/534,891, filed on Jan. 7, 2004.

(51) Int. Cl. C07H 21/04

(52) U.S. Cl. 536/25.4; 536/23.1

(57) ABSTRACT

Activatable ribozymal purification constructs are used to rapidly and efficiently purify RNA, including non-denatured RNA. The activatable ribozymal purification construct includes an activatable ribozyme covalently bound to a target RNA moiety. The activatable ribozyme is attached to an immobilizing moiety, which is capable of binding to solid support. Upon activation, the ribozyme cleaves the target RNA moiety thereby producing purified target RNA.

### CROSS-REFERENCES TO RELATED APPLICATIONS

This application claims the benefit of U.S. Provisional Application No. 60/534,891, filed Jan. 7, 2004, which is hereby incorporated by reference in its entirety for all purposes.

### BACKGROUND OF THE INVENTION

Discoveries of RNA interference (RNAi), small regulatory RNAs, and cis-acting RNA control elements highlight the central role RNA plays in gene expression. Furthermore, in the biotechnology sector RNA remains a focus for therapeutic design, including a new generation of antibiotics that bind the ribosomal RNA, and antiviral agents that target human immunodeficiency virus (HIV) and hepatitis C virus (HCV) RNAs, among others. To understand and to therapeutically exploit these diverse RNAs, we require a much deeper knowledge of RNA structure. Of particular importance are new tools to aid in the synthesis and purification of large quantities of RNA, as this remains a significant bottleneck in many structural efforts Doudna, Nat Struct Biol 7:954-956 (2000).

Transcription product RNAs are purified by preparative denaturing polyacrylamide gel electrophoresis, eluted from the gel matrix, concentrated, and refolded. Using this denaturing method, synthesis and purification of structural quantities of a single RNA sample (10-20 mg) typically requires one week and thus is not well suited to high-throughput. For many RNAs, significant time is spent optimizing refolding conditions to minimize unproductive conformations. Some well-known RNAs, such as E. coli tPNA^{Phe}, cannot be refolded into a conformationally homogeneous and active population (Uhlenbeck, Rna 1:4-6 (1995)). In some cases, this is overcome by a complex native purification involving a combination of anion exchange and gel filtration chromatography. Other RNA purification procedures have been developed, including those based on HPLC (Anderson et al., RNA 2:110-117 (1996); Shields et al., RNA 5:1259-1267 (1999)).

The present invention addresses these and other needs in the art of RNA purification.

### BRIEF SUMMARY OF THE INVENTION

It has been discovered that, surprisingly, RNA (including non-denatured RNA), may be rapidly and efficiently purified using an activatable ribozymal purification construct. This technique allows for rapid parallel purification of multiple RNA samples, and may be used with virtually any size or sequence of target RNA derived from both small (<1 mL) and large-scale (<10 mL) transcription reactions.

In a first aspect, the present invention provides an activatable ribozymal purification construct. The activatable ribozymal purification construct includes an activatable ribozyme covalently bound through a phosphodiester bond to a target RNA moiety. The activatable ribozyme is attached to an immobilizing moiety.

In another aspect, the present invention provides a method of purifying a target RNA. The method includes contacting an activatable ribozymal purification construct with a solid support to form an immobilized activatable ribozymal purification construct. The immobilized activatable ribozymal purification construct includes an activatable ribozyme covalently bound through a phosphodiester bond to a target RNA moiety. The activatable ribozyme is attached to an immobilizing moiety. The immobilizing moiety is attached to the solid support. The activatable ribozyme is then activated and allowed to cleave the phosphodiester bond between the activatable ribozyme and the target RNA moiety to form a mobilized target RNA. The mobilized target RNA is then separated from the activatable ribozyme thereby purifying the target RNA.

In another aspect, the present invention provides an expression vector including an expressible activatable ribozymal purification construct clone, a target RNA clone, and an RNA immobilizing moiety clone.

### BRIEF DESCRIPTION OF THE DRAWINGS

FIG. 1 illustrates a general scheme for the purification of any desired sequence (represented by RNA X, also referred to herein as the target RNA) using an activatable ribozymal construct of the present invention.

FIG. 2 is the DNA sequence of a cloning vector containing the activatable ribozymal purification construct clone pRAV4.

FIG. 3 is the DNA sequence of a cloning vector containing the activatable ribozymal purification construct clone pRAV12, where the asterisk (*) indicates the final nucleotide in the target RNA sequence, the activatable ribozyme cleavage site being at the phosphodiester bond on the 3′ side of the final ribonucleotide.

FIG. 4. illustrates the secondary structure of the activatable ribozymal purification construct derived from pRAV 12, with the location of the C75U mutation boxed.

FIG. 5 is an illustration of the purification of the T. maritima Ffh M domain (TmaM) as analyzed by a 15% SDS-PAGE gel with the lanes corresponding to: 1, cells prior to induction with 1 mM IPTG; 2, cells after induction with 1 mM IPTG; 3, supernatant fraction of the cell lysate; 4, fraction of protein eluted from the Ni²⁺-affinity column; 5, protein following cleavage with TEV protease; 6, peak fraction containing TmaM from the SP-sepaharose column; where the major band in each lane (except for lane 1) is TmaM.

FIG. 6 illustrates the test purification of RNA transcribed from the linearized pRAV4 vector, where the RNA was body-labeled using α-³²P-GTP during transcription, where an aliquot of the raw transcription reaction is shown on the left, and the pure product RNA is indicated.

FIG. 7 illustrates the diffraction pattern of crystals of the T. thermophila AC209P4P6 domain RNA that was transcribed and purified using an activatable ribozymal purification construct of the present invention.

FIG. 8 shows certain sequences of hepatitis delta virus activatable ribozymes having the “C75U” mutation (bold): A, the H6V activatable ribozyme employed in the pRAV4 plasmid conctruct; B, an H6V activatable ribozyme with a 5′ NgoMIV/NCO restriction site sequence as used in the pRAV12 plasmid construct; and C and D, certain conserved core sequences that contain the ribonucleotides essential for cleavage by these ribozyme.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described below are those well known and commonly employed in the art. Standard techniques are used for nucleic acid synthesis.

The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized.

Substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

As used herein, “nucleic acid” means either DNA, RNA, single-stranded, double-stranded, or more highly aggregated hybridization motifs, and any chemical modifications thereof, and includes ribonucleic acids. Modifications include, but are not limited to, those that provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping. The term “RNA” means ribonucleic acid. The term “nucleotide” means a subunit of a DNA or RNA including the nitrogenous base, one or more phosphates, and ribose or deoxyribose, and includes those nucleotides forming part of a DNA or RNA strand wherein the phosphate forms part of a covalent linkage with an adjacent nucleotide (e.g. phosphodiester linkage). A “ribonucleotide” is nucleotide with a ribose ring.

The use of the term “complementary” in relation to the binding of nucleic acids is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well-known in the art (see, e.g., Turner et al., CSH Symp. Quant. Biol. LII:123-133 (1987); Frier et al., Proc. Nat. Acad. Sci. 83:9373-9377 (1986); Turner et al., J. Am. Chem. Soc. 109:3783-3785 (1987). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

RNA of use in the present invention (e.g. ribozymes, target RNA, and RNA immobilizing moieties) include those RNAs with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions.

Where an activatable ribozymal purification construct “consists essentially of” an activatable ribozyme covalently bound through a phosphodiester bond to a target RNA moiety and an immobilizing moiety attached to the activatable ribozyme, other RNA sequences may be included that do not interfere with the operation or basic and novel characteristics of the activatable ribozymal purification construct. Likewise, where an activatable ribozyme “consists essentially of” a ribozymal catalytic core or specific nucleotide sequence, additional RNA sequences may be included on the 5′ end or the 3′ end of the ribozyme that do not interfere with the operation or basic and novel characteristics of the activatable ribozymal purification construct (e.g. sequences derived from restriction site sequences, linker sequences, ribozymal stabilizing sequences, immobilizing moiety stabilizing sequences).

Introduction

The present invention provides a completely new modality in the art of RNA purification. Novel compositions and methods are herein provided that allow for rapid and efficient purification of RNA, including RNA in its non-denatured state. Thus, RNA may be rapidly purified in its native folded confirmation.

I. Activatable Ribozymal Purification Constructs

In a first aspect, the present invention provides an activatable ribozymal purification construct. The activatable ribozymal purification construct includes an activatable ribozyme covalently bound through a phosphodiester bond to a target RNA moiety. The activatable ribozyme is attached to an immobilizing moiety. In some embodiments, the target RNA moiety is a non-denatured target RNA moiety.

An activatable ribozyme, as used herein, is a ribonucleic acid molecule capable of catalyzing the breaking of a phosphodiester bond within the activatable ribozymal purification construct when activated. The breaking of the phosphodiester bond (e.g. phosphotransesterification reaction) allows the target RNA moiety to be released from the activatable ribozymal purification construct, thereby facilitating purification of the target RNA.

Activatable ribozymes useful in the present invention have either no measurable catalytic activity in the absence of activation, or low catalytic activity in the absence of activation relative to the amount of activity after activation. In addition, the activatable ribozymes allow for facile separation of the target RNA moiety subsequent to catalytic cleavage. Thus, the activatable ribozymes have no sequence requirements upstream of the cleavage site that would prevent separation of the target RNA moiety.

In addition, the activatable ribozymes are capable, upon activation, of catalyzing the phosphodiester cleavage reaction to release the target RNA moiety within minutes or hours of activation. The activatable ribozymes of the present invention are further capable of catalyzing under conditions in which RNA is generally stable, which are well known in the art.

The activatable ribozymes of the present invention may be selected to minimize interference with binding of the immobilizing moiety with a solid support. The activatable ribozyme typically forms the active structure and is not susceptible to misfolding (e.g. due to adjacent RNA sequences). The activatable ribozymes may also cleave with high fidelity at a single site to release the target RNA moiety. In certain embodiments, the full-length target RNA moiety is released, rather than an attenuated target RNA moiety or a target RNA moiety having residual ribonucleic acids derived from the activatable ribozyme.

In some embodiments, the activatable ribozyme is less than 1000, 500, 100, or 50 nucleotides. The activatable ribozyme may catalyze cleavage at a temperature lower than 20° C., 21° C., 22° C., 23° C., 24° C., or 25° C. In other embodiments, the activatable ribozyme catalyzes complete cleavage of the target RNA moiety in less than 1 hour, less than 30 minutes, or less than 10 minutes. The activatable ribozyme may be readily engineered to contain convenient restriction sites, and readily interchanged with other activatable ribozymes.

Ribozymes useful in the present invention may be activated by effector molecules, physical signals, or combinations thereof. Physical signals include, but are not limited to, radiation (e.g. light radiation), temperature changes, pH changes, movement, physical conformational changes in samples, and combinations thereof. Thus, the invention includes those ribozymes that are activatable by effector molecules responsive to UV, IR, and/or visible light. See Koizumi et al., Nature Struct. Biol. 6, 1062-1071 (1999); and Grate et al., Proc. Nat. Acad. Sci. 96: 6131-6136 (1999) (disclosing a malachite green tagged RNA that cleaves upon activation with a laser). A wide variety of ribozymes that are catalytically activated using an effector molecule, whether alone or in combination with physical signals, are known in the art. Moreover, the rational design of ribozymes capable of being activated by specific effector molecules is well known. Such effector molecule activatable ribozymes, and methods for rationally designing the same, are discussed in detail, for example, in U.S. Pat. No. 6,630,306, which is hereby incorporated by reference in its entirety for all purposes. See also Winkler et al., Nature 428: 281-286 (2004); Koisumi et al., Nature Struct. Bio. 6: 1062-1071 (1999); and Seetherman et al., Nature Biotech. 19:336-341 (2001).

A vast number of activatable ribozymes with dynamic structural characteristics can be generated in a massively parallel fashion (Koizumi et al, Nature Struct. Biol. 6, 1062-1071 (1999)). New and highly-specific receptors have been made via in vitro selection (e.g. SELEX) using simple chromatographic and nucleic acid amplification techniques. RNA aptamers produced in this way can act as efficient and selective receptors for small organic compounds, metal ions, and even large proteins. New classes of aptamers may be isolated that are specific for innumerable compounds to create novel activatable ribozymes for use in the current inventions (e.g. activatable catalytic RNA aptamers). Using the guidance set forth above regarding characteristics of an activatable ribozyme and the examples set forth below, one of skill in the art may easily modify and test known or novel activatable ribozymes for use in the current invention. Thus, useful activatable ribozymes of the present invention include, for example, activatable hammerhead ribozymes, activatable hepatitis delta virus ribozymes, and activatable catalytic RNA aptamers (e.g. RNA aptamers modified to have catalytic activity upon activation and RNA aptamers coupled to activatable catalytic RNAs) that have the characteristics set forth above. See, for example, U.S. Pat. No. 6,630,306.

The activatable ribozyme must minimally include ribonucleic acids essential for catalytic functioning. This includes nucleotides involved directly in the chemistry of cleavage as well as nucleotides that form the structure of the ribozyme that is essential for function. In some embodiments, additional ribonucleotides may be added to the 5′ and/or 3′ end of this functioning ribozyme. The additional ribonucleotides may serve as linkers (also referred to herein as “linker sequences”) to the immobilizing moiety and/or the target RNA. The additional ribonucleotides may also serve to enhance catalytic function (e.g. stabilizing the structural conformation of the catalytic core). The additional ribonucleotides may also help in the stabilization of RNA immobilizing moieties. In certain embodiments, the sequence of ribonucleotides within the functioning ribozyme may be altered to add additional functionality to the construct. This includes restriction site sequences recognized by specific DNA-cleaving endonucleases. These sites are used in expression vectors to conveniently interchange activatable ribozymes for use in the activatable ribozymal purification constructs and to insert the DNA sequence encoding the target RNA. The altered ribonucleotides may also serve to enhance catalytic activity or help to stabilize the immobilizing moieties.

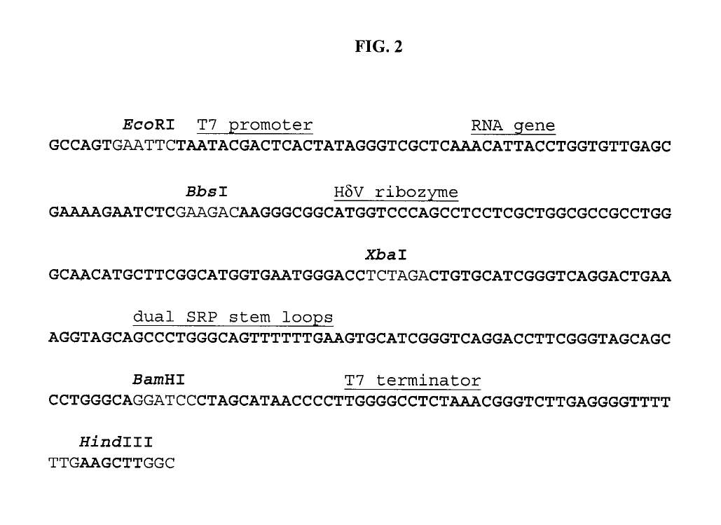

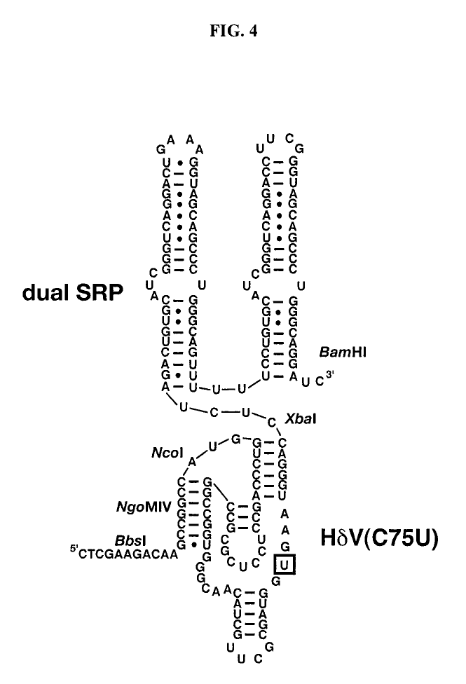

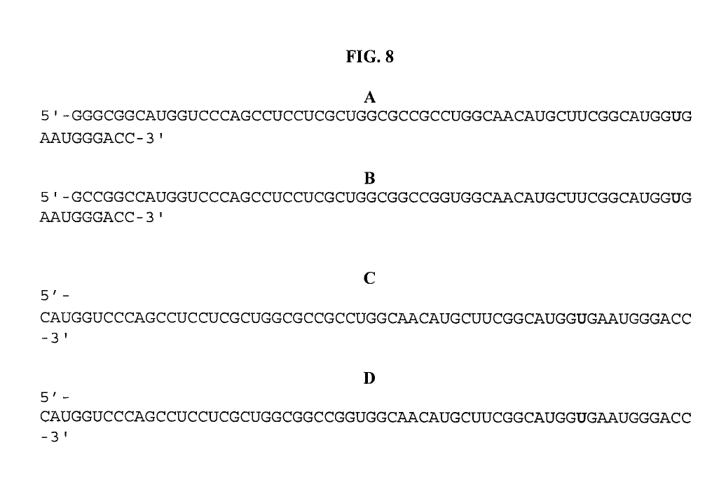

In some embodiments, the activatable ribozyme of the present invention is an activatable hepatitis delta virus ribozyme (H6V). Hepatitis delta virus ribozymes are well known in the art and are discussed in detail, for example, in Shih et al., Annual Review of Biochemistry 71: 887-917 (2002). An illustrative example of an activatable hepatitis delta virus ribozyme are those having a specific C to U mutation within a conserved region (generally known in the art as the “C75U” mutation), which is activated by the effector molecule, imidazole. See FIGS. 2, 3, 4, and 8; Perrotta et al., Science 286:123-126 (1999); Nishikawa et al., Eur J Biochem 269:5792-5803 (2002). Useful sequences of the HδV ribozyme are illustrated, for example, in FIGS. 4, 8A (an HδV activatable ribozyme) 8B (an HδV activatable ribozyme catalytic core with a 5′ NgoMIV/NCO restriction site sequence), and 8C and 8D (which contain the conserved core nucleotides essential for catalytic cleavage for these ribozymes).

An immobilizing moiety is a moiety capable of binding to a solid support covalently or non-covalently thereby attaching (also referred to herein as “immobilizing”) the immobilizing moiety to the solid support. The immobilizing moieties typically have high binding affinity and specificity for the solid support. In addition, the immobilizing moieties are selected to minimize chemical interference with ribozyme catalysis and degradation of RNA.

In some embodiments, the immobilizing moiety will bind to a complementary solid support. As used herein, a “complementary solid support” is a solid support having a binding moiety that specifically binds to the immobilizing moiety. A wide variety of immobilizing moieties are useful in the present invention.

In an exemplary embodiment, the immobilizing moiety includes a reactive group or half of an affinity tag binder pair (i.e an affinity tag or an affinity tag binder). For example, where the immobilizing moiety includes an affinity tag, the solid support to which the immobilizing moiety binds will include the complementary affinity tag binder, thereby forming an affinity tag-affinity tag binder pair. Likewise, where the immobilizing moiety includes a reactive group, the solid support may include a functional group to which the reactive group covalently binds thereby forming a reactive group-functional group pair.

A wide variety of affinity tag-affinity tag binder pairs are well known in the art and include, for example, those pairs having as one component of the pair: a signal recognition particle, U1A spliceosomal protein, MS2 coat protein, deiminobiotin, dethiobiotin, vicinal diol, digoxigenin, maltose, oligohistidine, glutathione, 2,4-dintrobenzene, phenylarsenate, ssDNA, dsDNA, ssRNA, polyhistidine, a hapten, T7 tag, S tag, H is tag, GST tag, PKA tag, HA tag, c-Myc tag, Trx tag, Hsv tag, CBD tag, Dsb tag, pelB/ompT, KSI, MBP tag, VSV-G tag, β-Gal tag, GFP tag, V5 epitope tag, or FLAG epitope tag (Eastman Kodak Co., Rochester, N.Y.). Affinity tag binders include moieties capable of specifically binding affinity tags, which are widely known in the art, such as the components above. Thus, the immobilizing moiety may be an affinity tag or an affinity tag binder, including one of the components disclosed above, or a moiety that is capable of specifically binding one of these components, such as a protein (e.g. antibody or antibody fragment) or nucleic acid (e.g. RNA aptamer, or complementary nucleic acid sequence).

In some embodiments, the immobilizing moiety includes a reactive group capable of covalently binding to the solid support. A wide variety of reactive groups are useful in the present invention. Currently favored classes of reactions are those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(i) epoxides, which can react with, for example, amines and hydroxyl compounds; and

(k) phosphoramidites and other standard functional groups useful in nucleic acid synthesis.

The reactive groups can be chosen such that they do not participate in, or interfere with, the reactions necessary to assemble the activatable ribozymal purification construct. Alternatively, a reactive group can be protected from participating in a reaction by the presence of a protecting group. Those of skill in the art will understand how to protect a particular reactive group from interfering with a chosen set of reaction conditions. For examples of useful protecting groups, See Greene et al., PROTECTIVE GROUPS IN ORGANIC SYNTHESIS, John Wiley & Sons, New York, 1991. Other useful covalent linkages may be found, for example, in texts relating to the art of solid phase synthesis of biomolecules such as peptides, polypeptide, proteins, nucleic acids and carbohydrates (see, e.g., Eckstein et al., Oligonucleotides and Analogues. A Practical Approach, (1991); Stewart et al., Solid Phase Peptide Synthesis, 2nd Ed., (1984); Seeberger, Solid support oligosaccharide synthesis and combinatorial carbohydrate libraries (2001)). In some embodiments, intein-mediated protein ligation may be used to attach the immobilizing reagent to a solid support (Mathys, et al., Gene 231:1-13, (1999); Evans, et al., Protein Science 7:2256-2264, (1998)).

Linkers may be employed to attach the reactive group to the remainder of the immobilizing moiety, or the functional groups to the remainder of the solid support. Linkers may include complementary chemical groups at the point of attachment to the remainder of the solid support or immobilizing moiety. Any appropriate linker may be used in the present invention, including those having a polyester backbone (e.g. polyethylene glycol), nucleic acid backbones, amino acid backbones, and derivatives thereof. A wide variety of useful linkers are commercially available (e.g. polyethylene glycol based linkers such as those available from Nektar, Inc. of Huntsville, Ala.).

Where the immobilizing moiety includes an affinity tag binder, an affinity tag, or a reactive group, the immobilizing moiety may be attached to the activatable ribozyme using any appropriate methods, including methods of covalent attachment and non-covalent attachment. For example, an immobilizing moiety may be attached to the activatable ribozyme though a complementary nucleic acid sequence (e.g. streptavidin chemically linked to a complementary nucleic acid sequence). Alternatively, a reactive group on the activatable ribozyme may be modified and covalently bound to the immobilizing moiety using a reactive group and/or linker (e.g. a modified streptavidin having a chemical moiety that covalently binds to the ribozymal reactive group). Reactive groups are discussed above in the context of immobilizing moiety attachment to the solid support and are equally applicable here to the attachment of an immobilizing moiety to the activatable ribozyme.

In other embodiments, the immobilizing moiety is an RNA that is covalently bound to the activatable ribozyme through a phosphodiester bond. For example, the target RNA, activatable RNA, and immobilizing moiety may be genetically engineered to a single linear RNA strand using molecular cloning techniques. See generally, Sambrook et al., Molecular Cloning: A laboratory manual, Cold Spring Harbor, N.Y.: Cold Spring Harbor Laboratory Press (2001); see also examples below and FIGS. 2, 3 and 4. Thus, in some embodiments, the target RNA is attached to the 5′-end of a single linear activatable ribozyme through a phosphodiester bond. An RNA immobilizing moiety may be attached to the 3′-end of a single linear activatable ribozyme through a phosphodiester bond.

The RNA immobilizing moiety may be an RNA aptamer capable of binding a protein or affinity tag on a solid support or an RNA sequence capable of hybridizing to a complementary sequence on the solid support. The RNA immobilizing moiety may be less than 500, 250, 100, or 50 nucleotides in length.

In an exemplary embodiment, the RNA immobilizing moiety is a protein-binding RNA immobilizing moiety, such as a signal recognition particle-binding RNA, a U1A spliceosomal protein binding RNA, or an MS2 coat protein-binding RNA. In other embodiments the RNA immobilizing moiety is an affinity tag-binding RNA immobilizing moiety, such as a streptavadin-binding RNA, or a polyadenosine RNA.

The immobilizing moiety and complementary solid support are typically chosen such that the immobilizing moiety/solid support interaction is readily disrupted (e.g. under non-denaturing conditions for RNA). In some embodiments, the immobilizing moiety and complementary solid support are chosen such that, after separation, no residual portion of the immobilizing moiety remains attached to the solid support, thereby regenerating the solid support after each use. Separation of the immobilizing moiety from the solid support may be accomplished by any appropriate means, such as contacting the immobilizing moiety/solid support with a denaturing liquid wash (e.g. ethanol or appropriately buffered aqueous solution) sufficient to disrupt the bonding interactions between the immobilizing moiety and the solid support. The selection of appropriate separation washes depends on the nature of the immobilizing moiety-solid support system chosen and are well within the abilities of a skilled artisan.

The term “solid support” refers to a material in the solid-phase that is capable of interacting with reagents in the liquid phase (e.g. in solution). Solid supports can be derivatized with reactive functional groups (such as those described above), affinity tags, affinity tag binders, biomolecules (including enzymes, peptides, oligonucleotides and polynucleotides), and the like. Typically, the solid support is a complementary solid support that includes a binding group or reactive group that is complementary to (i.e. binds to) an immobilizing moiety.

A wide array of solid supports are useful in the present invention. Solid supports are typically composed of insoluble materials. In some embodiments, the supports have a rigid or semi-rigid character, and may be any shape, e.g. spherical, as in beads, rectangular, irregular particles, resins, gels, microspheres, or substantially flat as in a microchip. Arrays of physically separate regions may be present on the support with, for example, wells, raised regions, dimples, pins, trenches, rods, pins, inner or outer walls of cylinders, and the like.

Preferred support materials include agarose, polyacrylamide, magnetic beads, polystyrene, controlled-pore-glass, polyacrylate hydroxethylmethacrylate, polyamide, polyethylene, polyethyleneoxy, or copolymers and grafts of such. The hydrophilic nature of the polyethyleneoxy groups promotes rapid kinetics and binding when aqueous solvents are used. Other solid supports include small particles, membranes, frits, non-porous surfaces, addressable arrays, vectors, plasmids, or polynucleotide-immobilizing media. Additionally, fullerenes may be used as a solid support, as well as derivatized fullerenes such as gadolinium fullerenes that contain paramagnetic properties.

In an exemplary embodiment, the solid support includes those composed of polystyrene, polyethylene, polyacrylamide, polypropylene, polyamide, Merrifield resin, sepharose, agarose, polystyrene, polydivinylbenzene, cellulose, alginic acid, chitosan, chitin, polystyrene-benzhydrylamine resin, an acrylic ester polymer, a lactic acid polymer, silica, silica gel, amino-functionalized silica gel, alumina, clay, zeolite, glass, controlled pore glass, or montmorillonite.

A target RNA is an RNA chosen to be purified using the methods and constructs of the present invention. A non-denatured target RNA moiety, mobilized non-denatured target RNA, and purified non-denatured target RNA, are RNAs of interest that have substantially retained their native and/or functional structure. The non-denatured target RNA has not been subjected to extracellular conditions that are known to disrupt intramolecular and/or intermolecular interactions leading to substantial unfolding or inactivation. One of skill in the art will immediately recognize that any appropriate sequence may be used. The target RNA and/or non-denatured target RNA may be less than is less than 5000, 2000, 1000, 500, 100, or 50 nucleotides.

II. Methods

In another aspect, the present invention provides a method of purifying a target RNA. The method includes contacting an activatable ribozymal purification construct with a solid support to form an immobilized activatable ribozymal purification construct. The immobilized activatable ribozymal purification construct includes an activatable ribozyme covalently bound through a phosphodiester bond to the target RNA moiety. The activatable ribozyme is attached to an immobilizing moiety. The immobilizing moiety is attached to the solid support. The activatable ribozyme is then activated and allowed to cleave the phosphodiester bond between the activatable ribozyme and the target RNA moiety to form a mobilized target RNA. The mobilized target RNA is then separated from the activatable ribozyme thereby purifying the target RNA.

In an exemplary embodiment, the target RNA is a non-denatured target RNA, the mobilized target RNA is a mobilized non-denatured target RNA, and the purified target RNA is a purified non-denatured target RNA.

In another exemplary embodiment, the method further includes separating the immobilizing moiety from the solid support thereby regenerating the solid support.

As disclosed above, the activatable ribozyme may be activated by any appropriate method, including methods employing effector molecules, physical signals, or combinations thereof. In certain embodiments, the activatable ribozyme is an effector molecule activated ribozyme. Where the activatable ribozyme is an effector molecule activated ribozyme, activation is accomplished by contacting the effector molecule activated ribozyme with an effector molecule. The effector molecule activated ribozyme is then allowed to cleave the phosphodiester bond between the activatable ribozyme and the target RNA moiety to form a mobilized target RNA, which is then separated from the activatable ribozyme thereby purifying the target RNA.

The activatable ribozyme may be contacted with the solid support in a liquid. Typically, the liquid is aqueous and is appropriately buffered so as to avoid denaturing the target RNA and degradation of the activatable ribozyme portions of the activatable ribozymal purification constructs. In some embodiments, the pH of the liquid may be adjusted to activate the activatable ribozyme. The optimal pH ranges of a wide variety of ribozymes are known in the art, and may be exploited to rationally design pH activatable ribozymes for use in the present invention. See Koizumi et al., Nat Struct Biol. 6:1062-1071 (1999).

In other embodiments, the activatable ribozyme is activated by contacting the activatable ribozymal purification construct with an effector molecule or an effector molecule and light. Exemplary methods of effector molecule and light activatable ribozymes are discussed in detail in Grate et al., Proc. Nat. Acad. Sci. 96: 6131-6136 (1999) and U.S. Pat. No. 6,630,306.

Properties of the activatable ribozymal purification construct, such as characteristics of immobilizing moieties, solid supports, activatable ribozymes, and target RNA moieties, are discussed in the previous section and are equally applicable to the methods provided herein.

A variety of effector molecules are useful in the present invention. Effector molecules are typically not present during transcription reactions. Effector molecules may be biological compounds, metal ions, or organic compounds. Examples of useful effector molecules include amino acids, amino acid derivatives, peptides (including peptide hormones), polypeptides, nucleosides, nucleotides, steroids, sugars or other carbohydrates, pharmaceuticals, theophylline, modified ATP, 3-methylxanthine, FMN, cobalt, cadmium, nickel, zinc, manganese, and substituted or unsubstituted heterocycle compounds (e.g. imidazole, pyridine, cytosine, and pyrazole). See U.S. Pat. No. 6,630,306. In certain embodiments, effector molecules have a molecular weight of about 300 Da or less.

In some embodiments, the effector molecule is a substituted or unsubstituted heteroaryl having a pKa from 5.5 to 8.5. In other embodiments, the effector molecule is a substituted or unsubstituted heteroaryl having a pKa from 6.5 to 7.5.

The effector molecule may be a substituted or unsubstituted imidazole, substituted or unsubstituted pyridine, substituted or unsubstituted pyrazole, or substituted or unsubstituted cytosine. The effector molecule may also be an unsubstituted imidazole, unsubstituted pyridine, unsubstituted pyrazole, or unsubstituted cytosine. In some embodiments, the effector molecule is unsubstituted imidazole.

III. Expression Vectors

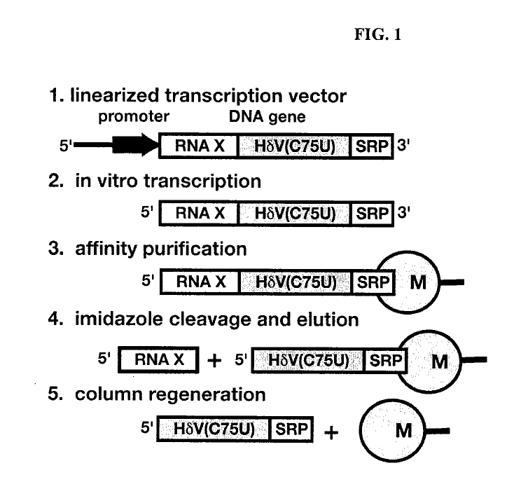

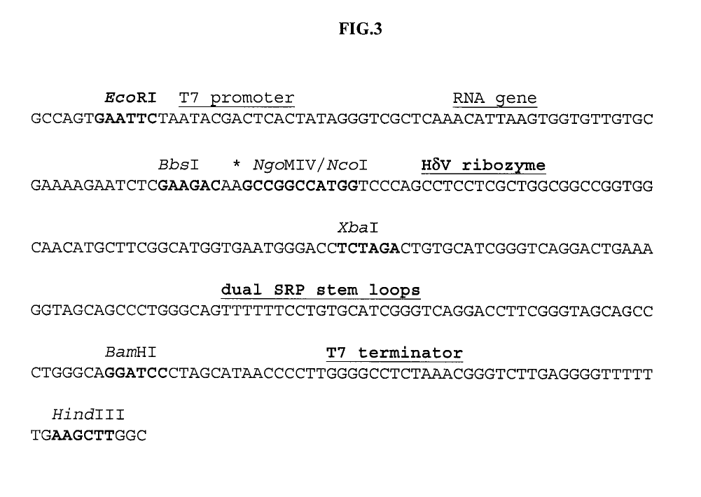

In another aspect, the present invention provides an expression vector including an expressible activatable ribozymal purification construct clone, a target RNA clone, and an RNA immobilizing moiety clone. The expression vector may include appropriate restriction site sequences (or other linker sequences) between the activatable ribozymal purification construct clone and the target RNA clone and/or the RNA immobilizing moiety clone. Appropriate restriction site sequences may also be included within the ribozyme or immobilization moiety sequences. The expression vector (e.g. an in vivo expression vector such as a plasmid) may be transcribed in vitro or transformed into an appropriate bacterial strain, such that the production of an activatable ribozymal purification construct can be induced (e.g. through the addition of IPTG). Following the growth and expression procedure, the host cells (e.g. bacteria host cell such as E. Coli) may be lysed and the lysate passed over solid support (e.g. an affinity column containing solid support that binds to the immobilizing moiety) to capture the expressed activatable ribozymal purification construct. Subsequent purification steps are disclosed above in the methods section. Thus, expression vectors capable of in vivo expression of activatable ribozymal purification construct allow extremely inexpensive production of target RNA, including non-denatured RNA, on a large scale. Examples of certain expression vectors containing an activatable ribozymal purification construct clone useful in producing in vivo expression vectors are discussed below and present in FIGS. 2 and 3. See also FIG. 1.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the activatable ribozymal purification constructs of the present invention are equally applicable to the methods of purification and expression vectors described herein. All publications, patents, and patent applications cited herein are hereby incorporated by reference in their entirety for all purposes.

### EXAMPLES

The following examples are provided merely to illustrate certain embodiments of the present invention. In the following examples, activatable ribozymal purification constructs are prepared and used to purify non-denatured target RNAs.

### Example 1

### Non-Denatured Target RNA Purification using Activatable Ribozymal Purification Constructs

A. General Description

The activatable ribozymal purification constructs exemplified below included an effector molecule activated ribozyme, the hepatitis delta virus (HδV) ribozyme. For this use, The HδV ribozyme sequence contained a C75U mutation that inactivates the ribozyme during the transcription reaction, but allowed for its own removal from the construct when contacted with the effector molecule, imidazole. See FIG. 3; Perrotta et al., Science 286:123-126 (1999); and Nishikawa et al., Eur J Biochem 269:5792-5803 (2002).

The immobilizing moiety included two tandem stem-loop motifs from the T. maritima SRP RNA that specifically and tightly bound the SRP protein, Ffh. This binding interaction was both thermodynamically robust and kinetically inert on the time scales of the purification procedure. The interaction of this RNA with its cognate protein is highly dependent on both pH and metal ion concentration, therefore the binding can be modulated with these two parameters. These two domains have been incorporated into high copy plasmid vectors (FIGS. 2-4) that allows for placement of the construct immediately downstream of any RNA sequence of interest.

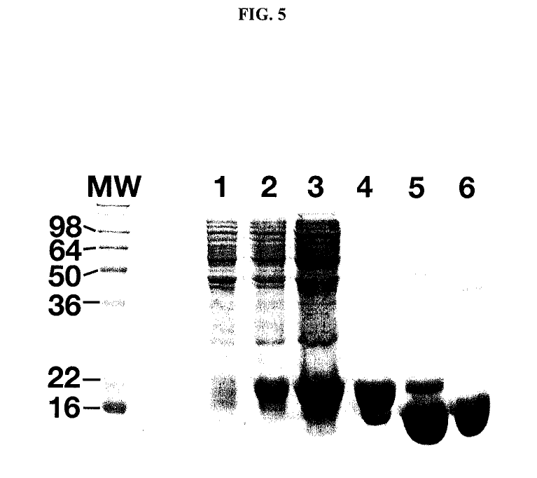

To create a chromatographic affinity matrix capable of specifically binding the above immobilizing moiety, the Thermotoga maritima SRP Ffh M-domain protein (referred to as TmaM) was coupled to a solid support matrix, the Affigel-10 matrix. This activated chromatographic media contains N-hydroxysuccinamide ester linked agarose, allowing covalent coupling of proteins through lysine residues. This protein-RNA complex is readily disrupted under non-denaturing conditions, gently regenerating the affinity matrix. TmaM is expressed in E. coli and purified in large quantities (˜70 mg/liter culture) with a straightforward purification protocol (FIG. 5), and ˜15 mg of protein is coupled to 1 mL of resin (corresponding to 1 μmol of potential RNA binding sites per mL resin) using established methods. See Prickett et al., Biotechniques 7:580-589 (1989); and Bardwell et al., Nucleic Acids Res 18:6587-6594 (1990).

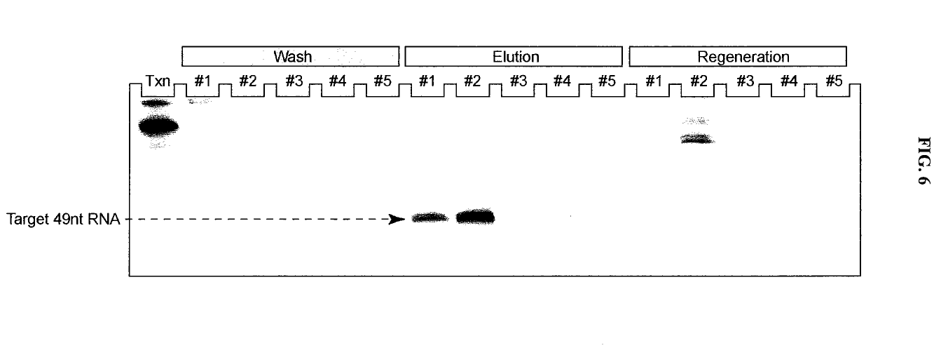

To test the purification scheme, a plasmid containing a 49-nt sequence from the plautia stali intestinal virus (PSIV) RNA was constructed (pRAV4, FIG. 3). See Sasaki et al., J Virol 73:1219-1226 (1999). A small (100 μL) two-hour transcription was performed, radioactively labeling the RNA during the reaction. The transcription reaction was diluted with load buffer, loaded directly onto M-domain affinity matrix, and washed (see materials and methods for buffer components). The product RNA was liberated from the column by adding imidazole-containing buffer, incubated for 2 hours, and collected by draining the column. Fractions (one column volume each) were desalted and analyzed on a denaturing polyacrylamide gel (FIG. 6).

Comparison of the raw transcription reaction with the wash fractions revealed almost quantitative uptake of the RNA construct, and virtually no leakage of RNA construct from the affinity column. Upon addition of imidazole, pure target RNA product was released. The non-denatured target RNA was the only species that was liberated from the column, as the activatable ribozyme and immobilizing moiety are retained on the column until treated with the regeneration buffer. Transcription and purification of the RNA shown in FIG. 6 required less than 5 hours.

The pRAV plasmids are completely modular with unique restriction sites defining each segment of the construct (FIGS. 2 and 3). Thus, besides cloning RNAs of interest, end users can easily make design changes that suit their particular applications.

B. Materials and Methods

1. Expression and Purification of T. maritima M Domain Protein

A domain of the Thermotoga maritima Ffh protein (TmaM) corresponding to amino acids 295-423 was cloned from genomic DNA (ATCC 43589) using a standard PCR reaction. See Sambrook et al. supra. The 5′ primer was designed to create a unique NcoI site at the 5′ end for cloning followed by sequences encoding a hexahistidine tag and a TEV protease cleavage site prior to the first amino acid of the M domain. The 3′ primer adds a second stop codon followed by a BamH1 site. Following amplification of a DNA fragment of the correct size and restriction digestion with NcoI/BamH1, it was ligated into pET15b (Novagen). The ligation reaction was transformed into DH5α cells, individual ampicillin resistant colonies isolated, plasmid DNA purified and the resulting vector (pTmaM4) sequence verified.

Expression of TmaM domain was performed by transforming the E. coli strain Rosetta(DE3)/pLysS (Novagen) with pTmaM4. These cells were grown in LB medium in eight 750 mL cultures at 37° C. to an absorbance (600 nm) of 0.7-0.8 and expression induced by the addition of 1 mM IPTG. The cultures were allowed to continue to grow for an additional 4-5 hours prior to harvesting by centrifugation. The cell pellets (each corresponding to 750 mL of culture) were immediately resuspended in Lysis Buffer (300 mM NaCl, 50 mM Tris-HCl, pH 8.0). Cell lysis was performed by three rounds of freeze/thaw in which the cells were frozen in liquid nitrogen and thawed to room temperature. The viscosity of the lysate was reduced by the addition of 20 units of DNase per liter cell growth (Boehringer Mannheim), 10 mM MgCl₂ and 10 mM CaCl₂ and incubated at 37° C. The cell lysate was clarified by centrifugation at 30,000×g for 30 minutes at 4° C. and the supernatant subjected to further purification.

TmaM domain was initially purified by passing the clarified lysate through a gravity column containing 20 mL of Ni²⁺-NTA affinity resin (QIAGEN). Following extensive washing with 300 mL of Wash Buffer (50 mM NaH₂PO₄, 300 mM NaCl, 20 mM imidazole, pH 8.0), the protein was eluted with Elution buffer (50 mM NaH₂PO₄, 300 mM NaCl, 250 mM imidazole, pH 8.0). Fractions containing the protein were pooled and cleaved with a 1:100 ratio of TEV protease TmaM domain overnight at room temperature (Lucast et al., Biotechniques 30:544-546, 548, 550 (2001)). The protein was exchanged into a buffer containing 100 mM NaCl, 10 mM Na-MES, pH 6.0 by dialysis in 6-8 kDa dialysis membrane and subsequently applied to an SP-sepharose column. Protein was eluted using a 0.1-1.5 M gradient of NaCl over a 300 mL volume; the protein eluted around 0.55 M NaCl. Fractions containing the protein were pooled, dialyzed into 50 mM K⁺-HEPES, pH 7.5. The concentration of the protein was assessed by absorbance at 280 nm using an extinction coefficient of 1615 M-1 cm⁻¹ and a molecular weight of 14,975 g/mol. The final yield of protein was 70 mg/L culture.

2. Preparation of TmaM4 Chromatographic Matrix

TmaM4 was covalently coupled to an activated support, Affigel-10 (BioRad), according to the protocol supplied. 25 mL of beads were washed with 250 mL of ice-cold ddH₂O by vacuum filtration without allowing the beads to completely dry out during the procedure. The beads were then added to 50 mL of a 550 μM protein solution and allowed to incubate at 4° C. for 2 hours and room temperature for 5 hours with gentle agitation. After coupling, the supernatant containing unreacted protein was removed by placing the slurry in a 20×2.5 cm Econo-column (BioRad). The coupled resin washed with 2×50 mL aliquots of 50 mM K⁺-HEPES, pH 7.5 followed by 50 mL of 50 mM Tris-HCl, pH 8.0. To block unreacted N-hydroxysuccinamide groups, the column was allowed to incubate overnight in Tris buffer at 4° C. The resin was finally washed and stored in a buffer containing 200 mM NaCl, 10 mM MgCl₂, 50 mM Tris-HCl, pH 8.0 and 0.1% Na-azide and stored at 4° C.

3. Construction of an Activatable Ribozymal Purification Construct Expression Vector

Standard PCR and cloning strategies were used to create a DNA insert that contains a T7 RNA polymerase promoter, a 49 nucleotide insert (nt 6157-6195) of the plautia stali intestinal virus IRES RNA, the C75U mutant genomic HδV ribozyme, two T. maritima SRP RNA stem loops and a T7 terminator (FIG. 2). The resulting DNA fragment was digested with EcoR1 and HindIII, ligated into pUC19, and the reaction transformed into E. coli DH5 cc cells. Individual ampicillin resistant colonies were picked, miniprepped and sequence verified; the resultant plasmid is subsequently referred to as pRAV4 (RAV=RNA Affinity Vector). This plasmid was used in the test purification of FIG. 5 and as the basis for further optimization and modification.

Modifications of this plasmid were subsequently made using oligonucleotide mediated site directed mutagenesis. The first change involves adding three Watson-Crick base pairs to the second SRP stem-loop to stabilize the terminal helix. A further alteration was made to the first helix of the H6V ribozyme to add NgoMIV and NcoI restriction sites to facilitate cloning into this vector. The resultant plasmid was sequence verified (sequence of insert shown in FIG. 3) and is referred to as pRAV12.

4. In Vitro Transcription of RNA

RNA was transcribed in vitro from linearized plasmid DNA or directly from PCR products using established protocols. For reactions from plasmid DNA, the plasmid was linearized with BamH1 and used in in vitro transcription reactions at a final concentration of 75 μg/ml. For reactions from PCR products, the reactions were prepared using the Qiagen PCR clean-up kit. Reactions consisted of 30 mM Tris-HCl pH 8.0, 10 mM DTT, 0.1% Triton X-100, 0.1 mM spermidine-HCl 8 mM each NTP (Sigma, pH adjusted to 8.0), 40 mM MgCl₂, 50 μg/mL T7 RNA polymerase, and 1 unit/mL inorganic pyrophosphatase (Sigma), and template DNA at 75 μg/mL. Reactions were incubated for 1.5 to 2 hours (or as indicated in the figures) at 37° C.

5. Insertion of the AC209 variant of the T. thermophila Group I intron P4P6 domain into the Activatable Ribozymal Purification Construct Expression Vector

A gene corresponding to the (Δ209)P4P6 domain was cloned using a nested PCR strategy. The gene was amplified with two inner primers (5′ primer, TAATACGACTCACTATAGGAATTGCGGGAAAGGGGT; 3′ primer, CGGGCGGAAGACGCGCCCTGAACTGCATCCATATCA) and two outer primers (5′ primer, GCGCGCGAATTCTAATACGACTCACTATAG; 3′ primer, CCGCGGGCGGAAGACGCGCCC). The resulting product was restriction digested with EcoRI and BbsI, ligated into pRAV4 digested with the same enzymes, and transformed into E. coli DH5α cells. Individual ampicillin resistant colonies were picked and screened for the presence of the mutant P4P6 insert. A single isolate containing the proper insert, pR4P4P6, was subsequently prepared from 1.5 L of culture to obtain sufficient material for large scale in vitro transcription reactions. Purified vector was linearized with BamH1, extracted twice with an equal volume of 25:24:1 phenol (pH 8.1):chloroform:isoamyl alcohol, ethanol precipitated and brought up in sufficient 1× T.E. buffer to yield a working stock of ˜1 mg/mL.

6. Transcription and Purification of the T. thermophila Group I Intron P4P6 Domain

The (ΔC209)P4P6-construct was transcribed using standard conditions (see above) in a 10 mL reaction mixture for 1.5 hours at 37° C. Following the completion of transcription, 25 mL of Column Wash Buffer (25 mM Tris-HCl, pH 8.0, 250 mM NaCl, 10 mM MgCl₂) was added to the reaction and applied to 15 mL of affinity resin in a 20×2.5 cm glass column (Econo-column, BioRad).

Binding of the RNA to the column was affected by passing the diluted transcription reaction through the column four times with a flow rate of approximately 2.0 mL/min at room temperature. Following the last reapplication of the flow-through, the column washed with 5×30 mL aliquots of Wash Buffer at a flow rate of 3.5 mL/min. After the last wash, 20 mL of Wash Buffer plus 200 mM imidazole (pH 8.0) was added and allowed to pass through the column. At this point, the column was stopped, another 20 mL aliquot of Cleavage Buffer was added and allowed to incubate at 37° C. for 2 hours to facilitate removal of the P4P6 domain RNA from the construct. Non-denatured target RNA product was recovered by opening the column and collecting 5×20 mL aliquots; product appeared to be completely eluted by the second fraction, as judged by an ethidium bromide-stained 8% denaturing polyacrylamide gel. The chromatographic matrix was regenerated by removal of the cleaved and uncleaved immobilizing moiety by applying 5×50 mL aliquots of Regeneration Buffer (1.0 M LiCl, 25 mM Na₂EDTA, 174 mM glacial acetic acid). Immediately following regeneration, the column was re-equilibrated in Wash Buffer+0.1% Na-azide and stored at 4

7. Crystallization of Purified T. Thermophila P4P6 domain.

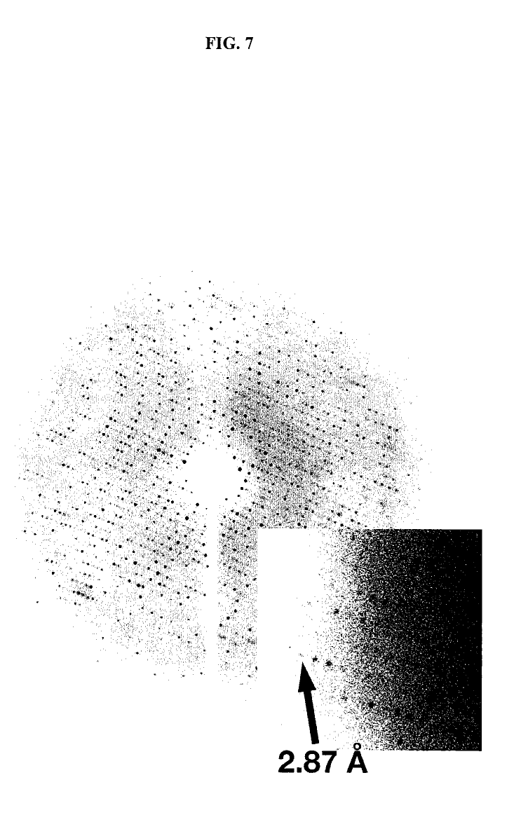

To demonstrate that this method generates high-quality RNA, we purified the AC209 mutant of the T. thermophila group I intron P4P6 domain using the activatable ribozymal purification construct and crystallized the purified product. The purified RNA readily crystallizes under a broad range of conditions. These crystals diffract synchrotron x-ray radiation to 2.2 Å resolution (Juneau et al., Structure (Camb) 9:221-231 (2001)). P4P6 domain RNA was purified from a 10 mL transcription reaction and then concurrently the RNA concentrated and the buffer exchanged in a centrifugal filter device. At no point prior to setting up crystallization drops was the RNA denatured. Single crystals grew in previously reported conditions (Juneau K, Podell E, Harrington DJ, Cech TR. 2001. Structural basis of the enhanced stability of a mutant ribozyme domain and a detailed view of RNA—solvent interactions. (Juneau et al., Structure (Camb) 9:221-231 (2001)) (FIG. 7) as well as in condition #5 of a commercially available sparse matrix screening kit (Scott et al., J Mol Biol 250:327-332 (1995)). These crystals diffract to ˜2.8 Å resolution using a rotating anode home x-ray source (I/σ=2.1 for the 2.93-2.80 Å resolution bin) (FIG. 7). The space group is P2₁2₁2₁ with unit cell dimensions of a=75.4 Å, b=125.8 Å and c=145.5 Å, values very close to those reported (Juneau et al., Structure (Camb) 9:221-231 (2001)). Structural basis of the enhanced stability of a mutant ribozyme domain and a detailed view of RNA—solvent interactions. (Juneau et al., Structure (Camb) 9:221-231 (2001)). Furthermore, the mosaicity of these crystals is 0.45° on the home source, which is as good, if not better, than crystals of the same RNA purified using traditional techniques (E. Podell, personal communication).

To prepare the RNA for crystallization, the two elution fractions containing the product were pooled and concentrated using a centrifuge concentrator (Amicon, Ultra) with a 10,000 MWCO. After concentration down to a volume of 500 μL, the sample was exchanged into a buffer containing 10 mM NaCl, 25 mM MgCl₂, 5 mM K⁺-HEPES, pH 7.5 with three exchanges against 15 mL of buffer. The final concentration of the RNA stock used for crystallization trials was 5.0 mg/mL as determined using the calculated extinction coefficient based upon the nucleotide sequence. This RNA was tested for crystallizability with a highly successful nucleic acid-oriented sparse matrix (Natrix, Hampton) using the hanging drop method and mixing 2 μL of the RNA solution with 2 μL of the appropriate mother liquor and incubated at 20° C. Crystallization of the mutant P4P6 domain was also effected using the exact conditions described by Juneau et al, except that the RNA was not heat annealed prior to setting up drops (Juneau et al., Structure (Camb) 9:221-231 (2001)).

In order to assess their quality, the crystals were cryoprotected for 1 hour in a buffer described by Juneau et al and flash frozen in liquid nitrogen (Juneau et al., Structure (Camb) 9:221-231 (2001)). Data was collected on a R-AXIS IV++instrument with CuKα x-ray radiation using 0.50 oscillation angle and five minute exposures. A 25° wedge of data was reduced with D*TREK.

### Example 2

### Immobilizing Moieties

In theory, any immobilizing moiety could be used with this protocol, including commercially available matrices. We explored two other immobilizing moieties: a 15 nucleotide poly-A immobilizing moiety that binds poly-dT resin and a 3 tandem repeat Sephadex G-100 aptamer (Srisawat et al. Rna 7:632-641 (2001); Srisawat et al., Nucleic Acids Res 29:E4 (2001)). Both contained the HδV C75U activatable ribozyme 5′ of the immobilizing moiety.

The poly-A immobilizing moiety coupled poorly to the column, with unacceptably high amounts of the transcribed material passing through the matrix (data not shown). Thus, where poly-A immobilizing moieties and poly-dT resins are employed, it is recommended that higher salt concentrations and poly-A/poly-dT nucleic acids longer than 15 bases be employed.

The Sephadex aptamer immobilizing moiety slowly released from the column during the wash and elution steps, leading to contamination of the target non-denatured RNA (data not shown). To improve this system, it is suggested that the immobilizing moieties be mutated to improve the kinetics of the binding reaction (e.g. using SELEX). Alternatively or in addition, faster activatable ribozymes may be used to minimize contamination.

The commonly used U1A and MS2 coat protein-RNA interactions could be used in place of the TmaM-RNA interaction, with the appropriate RNA element placed between the XbaI and BamHI sites (FIG. 3). This capability further generalizes the method to RNAs whose purification is incompatible with the TmaM-SRP RNA interaction (for example, SRP RNAs).

### Example 3

### Hammerhead Ribozymes as Activatable Ribozyme

A common method in RNA transcription is to use a hammerhead ribozyme at the 5′ end of the transcript. This provides a number of distinct benefits: chemical homogeneity at the 5′ terminus of the desired product, the use of a strong initiation sequence at the 5′ end of the transcript, and the lack of sequence requirements at the 5′ end of the product RNA.

In some embodiments, the activatable ribozymal purification construct accommodates a 5′ hammerhead ribozyme where the number of base pairs between the hammerhead activatable ribozyme and the non-denatured target RNA is limited (e.g. 3-4 bps), allowing the cleaved hammerhead ribozyme to efficiently dissociate from the product non-denatured target RNA. For example, is has been observed that only 1 base pair is needed to induce hammerhead ribozymal cleavage.

What is claimed is:

1. An activatable ribozymal purification construct comprising an activatable ribozyme covalently bound through a phosphodiester bond to a target RNA moiety, wherein the activatable ribozyme is attached to an immobilizing moiety.

2. The activatable ribozymal purification construct of claim 1, wherein said target RNA moiety is a non-denatured target RNA moiety.

3. The activatable ribozymal purification construct of claim 1, wherein the immobilizing moiety is an RNA immobilizing moiety.

4. The activatable ribozymal purification construct of claim 3, wherein the immobilizing moiety is covalently bound to the activatable ribozyme.

5. The activatable ribozymal purification construct of claim 4, wherein said immobilizing moiety is a protein-binding RNA.

6. The activatable ribozymal purification construct of claim 4, wherein said immobilizing moiety is a signal recognition particle-binding RNA, a U1A spliceosomal protein binding RNA, an MS2 coat protein-binding RNA, a streptavadin-binding RNA, or a polyadenosine RNA.

7. The activatable ribozymal purification construct of claim 1, wherein said activatable ribozyme is an activatable ribozyme, light activatable ribozyme, or pH activatable ribozyme.

8. The activatable ribozymal purification construct of claim 7, wherein said activatable ribozyme is an activatable hepatitis delta virus ribozyme, or a hammerhead ribozyme.

9. The activatable ribozymal purification construct of claim 7, wherein said activatable ribozyme is an activatable hepatitis delta virus ribozyme.

10. The activatable ribozymal purification construct of claim 9, wherein said activatable hepatitis delta virus ribozyme consists essentially of the ribonucleic acid sequence of FIG. 8C or FIG. 8D.

11. The activatable ribozymal purification construct of claim 1, wherein said covalent bond is a phosphodiester bond.

12. A method of purifying a target RNA, said method comprising the steps of:
(a) contacting an activatable ribozymal purification construct with a solid support to form an immobilized activatable ribozymal purification construct, said immobilized activatable ribozymal purification construct comprising an activatable ribozyme covalently bound through a phosphodiester bond to a target RNA moiety, wherein the activatable ribozyme is attached to an immobilizing moiety, and said immobilizing moiety is attached to said solid support;
(b) activating said activatable ribozyme;
(c) after step (b), allowing said activatable ribozyme to cleave the phosphodiester bond between the activatable ribozyme and the target RNA moiety to form a mobilized target RNA;
(d) separating said mobilized target RNA from the activatable ribozyme thereby purifying said target RNA.

13. The method of claim 12, wherein said target RNA moiety is a non-denatured target RNA moiety, said mobilized target RNA is a mobilized non-denatured target RNA, and said target RNA is a non-denatured target RNA.

14. The method of claim 12, further comprising, (e) separating the immobilizing moiety from the solid support thereby regenerating the solid support.

15. The method of claim 12, wherein said activatable ribozyme is activated by contacting said activatable ribozymal purification construct with an effector molecule.

16. The method of claim 12, wherein said activatable ribozyme is contacted with said solid support in a liquid.

17. The method of claim 16, wherein said activatable ribozyme is activated by adjusting the pH of the liquid.

18. The method of claim 12, wherein said activatable ribozyme is activated by contacting said activatable ribozymal purification construct with an effector molecule and light.

19. The method of claim 12, wherein the immobilizing moiety is an RNA immobilizing moiety.

20. The method of claim 19, wherein the immobilizing moiety is covalently bound to the activatable ribozyme.

21. The method of claim 20, wherein said immobilizing moiety is a protein-binding RNA.

22. The method of claim 19, wherein said RNA immobilizing moiety is a signal recognition particle-binding RNA, a U1A spliceosomal protein binding RNA, an MS2 coat protein-binding RNA, a streptavadin-binding RNA, or a polyadenosine RNA.

23. The method of claim 15, wherein said activatable ribozyme is an activatable hammerhead ribozyme.

24. The method of claim 23, wherein said hammerhead ribozyme forms no more than 4 base pairs with the mobilized target RNA.

25. The method of claim 23, wherein said activatable hammerhead ribozyme is an activatable hepatitis delta virus ribozyme.

26. The method of claim 25, wherein said activatable hepatitis delta virus ribozyme consists essentially of the ribonucleic acid sequence of FIG. 8C or FIG. 8D.

27. The method of claim 12, wherein the immobilizing moiety is attached to the activatable ribozyme via a phosphodiester bond.

28. The method of claim 15, wherein said effector molecule is a substituted or unsubstituted heteroaryl having a pKa from 5.5 to 8.5.

29. The method of claim 15 wherein said effector molecule is a substituted or unsubstituted heteroaryl having a pKa from 6.5 to 7.5.

30. The method of claim 15, wherein said effector molecule is substituted or unsubstituted imidazole, substituted or unsubstituted pyridine, substituted or unsubstituted pyrazole, or substituted or unsubstituted cytosine.

31. The method of claim 15, wherein said effector molecule is unsubstituted imidazole, unsubstituted pyridine, unsubstituted pyrazole, or unsubstituted cytosine.

32. The method of claim 15, wherein said effector molecule is unsubstituted imidazole.
